(19) United States
(12) Patent Application Publication
(10) Pub. No.: US 2010/0330018 A1
(43) Pub. Date: Dec. 30, 2010

(54) COMPOSITION CONTAINING A SUPERABSORBENT POLYMER AND AN ORGANIC UV SCREENING AGENT

(75) Inventors: Raluca LORANT, Thiais (FR); Laure Fageon, Paris (FR); Karl Boutelet, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/813,564

(22) Filed: Jun. 11, 2010

Related U.S. Application Data
(60) Provisional application No. 61/224,493, filed on Jul. 10, 2009.

(30) Foreign Application Priority Data
Jun. 24, 2009 (FR) 09 54311

(51) Int. Cl. A61K 8/72; A61Q 5/00

(52) U.S. Cl. 424/70.9

(57) ABSTRACT

A composition in the form of an emulsion containing an aqueous phase, a fatty phase, a superabsorbent polymer, and an organic UV screening agent. Method for the treatment of a keratinous substance whereby the composition described is applied to the keratinous substance.

### REFERENCE TO PRIOR APPLICATIONS

This application claims priority to U.S. provisional application Ser. No. 61/224,493, filed Jul. 10, 2009; and to French patent application 09 54311, filed Jun. 24, 2009, both incorporated herein by reference.

### FIELD OF THE INVENTION

The present patent application relates to a composition in the form of an emulsion comprising a superabsorbent polymer and an organic UV screening agent, and to the use of the composition in the cosmetic and dermatological fields, in particular for caring for or treating keratinous substances.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. In this regard, the description herein is to be understood as illustrative in nature, and not as restrictive.

### BACKGROUND OF THE INVENTION

Antisun compositions are often provided in the form of an emulsion of oil-in-water or water-in-oil type, of gels or of products which are anhydrous and which comprise, at various concentrations, one or more lipophilic and/or hydrophilic, insoluble and/or soluble and organic and/or inorganic screening agents capable of selectively absorbing harmful UV radiation, these screening agents and their amounts being selected as a function of the protection factor desired. According to their lipophilic nature or, on the contrary, their hydrophilic nature, these screening agents can be distributed, respectively, either in the fatty phase or in the aqueous phase of the final composition.

Organic screening agents are commonly used in antisun formulations. However, for a number of them, in particular lipophilic screening agents, their incorporation in emulsions brings about a decline in the cosmetic feel, in particular a tacky effect during application to the skin and then after penetration of the product. This tacky effect makes the use of the product unpleasant for the user and this unpleasantness is all the more pronounced when these products are used in times of great heat.

In order to overcome the problems of tackiness of cosmetic compositions comprising lipophilic active principles possessing sugar units, silicone elastomers, which make it possible to reduce the tacky effect, have been proposed. However, this solution cannot be envisaged in the case of fat-soluble screening agents due to their incompatibility with silicone compounds.

### DETAILED DESCRIPTION OF THE PREFERRED EMBODIMENTS

One object of the invention is to be able to produce emulsions exhibiting good cosmetic properties without having the disadvantages of the prior art.

The inventors have discovered that the use of a superabsorbent polymer in combination with an organic UV screening agent makes it possible to reduce, even eliminate, the tacky effect and makes it possible in particular to produce cosmetic compositions and in particular emulsions having very good cosmetic properties, especially of softness and of slip, on application to the skin, even in the presence of a high level of lipophilic UV screening agents.

A subject-matter of the present invention is thus a composition in the form of an emulsion comprising at least one aqueous phase and at least one fatty phase, a superabsorbent polymer and at least one organic UV screening agent.

As the composition of the invention is intended in particular for a topical application, it preferably comprises a physiologically acceptable medium, that is to say a medium compatible with all keratinous substances, such as the skin, nails, mucus membranes and keratinous fibres (such as hair or eyelashes).

The composition obtained according to the invention preferably exhibits the advantage of having a texture which is homogeneous, very soft, non-tacky and fresh on application and thus very pleasant to use.

Another subject-matter of the invention is a method for the cosmetic treatment of keratinous substances which comprises applying, to the keratinous substance, a composition as defined above.

### Superabsorbent Polymer

The term “superabsorbent polymer” is understood to mean a polymer which is capable, in its dry state, of spontaneously absorbing at least 20 times its own weight of aqueous fluid, in particular of water and especially of distilled water. Such superabsorbent polymers are described in the work “Absorbent Polymer Technology, Studies in Polymer Science 8” by L. Brannon-Pappas and R. Harland, published by Elsevier, 1990.

These polymers have a high capacity for absorbing and retaining water and aqueous fluids. After absorption of the aqueous liquid, the particles of the polymer thus impregnated with aqueous fluid remain insoluble in the aqueous fluid and thus retain their separated particulate state.

The superabsorbent polymer can have a water-absorbing capacity ranging from 20 to 2000 times its own weight (i.e., 20 g to 2000 g of water absorbed per gram of absorbent polymer), preferably from 30 to 1500 times and better still ranging from 50 to 1000 times. These water-absorbing characteristics are defined at standard temperature (25° C.) and pressure (760 mm Hg, i.e. 100 000 Pa) conditions and for distilled water. The value of the water-absorbing capacity of a polymer can be determined by dispersing 0.5 g of polymer(s) in 150 g of a water solution, by waiting 20 minutes, by filtering the nonabsorbed solution through a 150 μm filter for 20 minutes and by weighing the nonabsorbed water.

The superabsorbent polymer used in the composition of the invention is preferably provided in the form of particles which, once hydrated, swell with the formation of soft beads having a number-average diameter of 10 μm to 1000 μm.

Preferably, the superabsorbent polymer exhibits a number-average size of less than or equal to 100 μm, preferably of less than or equal to 50 μm, for example ranging from 10 μm to 100 μm.

Preferably, the superabsorbent polymers used in the present invention are provided in the form of spherical particles.

The superabsorbent polymers used in the present invention are preferably crosslinked acrylic homo- or copolymers which are preferably neutralized and which are provided in the particulate form.

Mention may in particular be made of the polymers chosen from:

crosslinked sodium polyacrylates, such as, for example, those sold under the names Octacare X100, X110 and RM100 by Avecia, those sold under the names Flocare GB300 and Flosorb 500 by SNF, those sold under the names Luquasorb 1003, Luquasorb 1010, Luquasorb 1280 and Luquasorb 1100 by BASF, those sold under the names Water Lock G400 and G430 (INCI name: Acrylamide/Sodium Acrylate Copolymer) by Grain Processing, or Aqua Keep 10 SH NF, provided by Sumitomo Seika,

starches grafted by an acrylic polymer (homopolymer or copolymer) and in particular by sodium polyacrylate, such as those sold under the names Sanfresh ST-100C, ST100MC and IM-300MC by Sanyo Chemical Industries (INCI name: Sodium Polyacrylate Starch),

hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing (INCI name: Starch/Acrylamide/Sodium Acrylate Copolymer),

polymers based on starch, on gum and on cellulose derivative, such as those comprising starch, guar gum and sodium carboxymethyl cellulose, sold under the name Lysorb 220 by Lysac,

and their blends.

Preferably, the superabsorbent polymer is chosen from crosslinked sodium polyacrylates, preferably in the form of particles having a number-average size (or mean diameter) of less than or equal to 100 microns, more preferably in the form of spherical particles. These polymers preferably have a water-absorbing capacity of 10 to 100 g/g, preferably of 20 to 80 g/g and better still of 50 to 70 g/g.

The superabsorbent polymer can be present in the composition of the invention in a content as active material ranging, for example, from 0.03 to 15% by weight, preferably from 0.05 to 10% by weight, preferably from 0.1 to 5% by weight, preferentially from 0.1 to 3% by weight, indeed even from 0.1 to 2% by weight, with respect to the total weight of the composition.

### Organic Screening Agents

The organic screening agents are generally present in the compositions according to the invention in proportions ranging from 0.05 to 30% by weight, with respect to the total weight of the composition, preferably ranging from 0.1 to 20% by weight and better still ranging from 0.5 to 15% by weight, with respect to the total weight of the composition.

The organic screening agents can be chosen from lipophilic or hydrophilic organic screening agents or their mixtures.

The term “lipophilic screening agent” is understood to mean any screening agent capable of being completely dissolved in the molecular state in a liquid fatty phase or else of being dissolved in the colloidal form (for example in the micelle form) in a liquid fatty phase.

The term “hydrophilic UV screening agent” is understood to mean any agent which screens out UV radiation and which is capable of being completely dissolved in the molecular state in the aqueous phase of the emulsion or else of being dissolved in the colloidal form (for example in the micelle form) in a aqueous phase of the emulsion.

Preferably, the composition comprises at least one lipophilic organic screening agent.

The lipophilic organic screening agents can be chosen from para-aminobenzoic acid derivatives, salicylic derivatives, cinnamic derivatives, benzophenones or aminobenzophenones, anthranilic derivatives, dibenzoylmethane derivatives, ββ-diphenylacrylate derivatives, benzylidenecamphor derivatives, phenylbenzimidazole derivatives, benzotriazole derivatives, triazine derivatives, bisresorcinyltriazines, imidazoline derivatives, benzalmalonate derivatives, 4,4-diarylbutadiene derivatives, benzoxazole derivatives, merocyanines, diphenylbutadienemalonate or -malonitrile derivatives, chalcones and their mixtures.

Mention may be made, among lipophilic UV-A screening agents capable of absorbing UV radiation from 320 to 400 nm, of:

### Dibenzoylmethane Derivatives:

4-isopropyldibenzoylmethane, sold under the name of “Eusolex 8020” by Merck, which corresponds to the following formula:

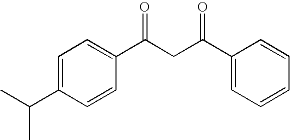

1-(4-methoxy-1-benzofuran-5-yl)-3-phenylpropane-1,3-dione, provided for sale by Quest under the name of Pongamol, of formula:

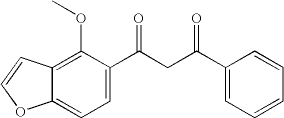

1-(4-(tert-butyl)phenyl)-3-(2-hydroxyphenyl)propane-1,3-dione, of formula:

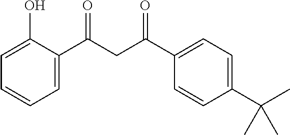

Butyl Methoxydibenzoylmethane, sold in particular under the trade name “Parsol 1789” by Hoffmann-La Roche,

### Aminobenzophenones:

n-Hexyl 2-(4-diethylamino-2-hydroxybenzoyl)benzoate, sold under the trade name “Uvinul A+”,

Anthranilic Derivatives:

Menthyl anthranilate, sold under the trade name “Neo Heliopan MA” by Haarmann and Reimer,

### 4,4-Diarylbutadiene Derivatives:

1,1-Dicarboxy-(2,2′-dimethylpropyl)-4,4-diphenylbutadiene.

Mention may be made, among lipophilic UV-B screening agents capable of absorbing UV radiation from 280 to 320 nm, of:

para-Aminobenzoates:
Ethyl PABA
Ethyl Dihydroxypropyl PABA
Ethylhexyl Dimethyl PABA (Escalol 507 from ISP)

### Salicylic Derivatives:

Homosalate, sold under the name “Eusolex HMS” by Rona/EM Industries,
Ethylhexyl Salicylate, sold under the name “Neo Heliopan OS” by Haarmann and Reimer,
Dipropylene Glycol Salicylate, sold under the name “Dipsal” by Scher,
TEA Salicylate, sold under the name “Neo Heliopan TS” by Haarmann and Reimer,

### Cinnamates:

Ethylhexyl Methoxycinnamate, sold in particular under the trade name “Parsol MCX” by Hoffmann-La Roche,

Isopropyl Methoxycinnamate,

Isoamyl Methoxycinnamate, sold under the trade name “Neo Heliopan E 1000” by Haarmann and Reimer,

Diisopropyl Methylcinnamate,

Cinoxate,

Glyceryl Ethylhexanoate Dimethoxycinnamate,

### β,β-Diphenylacrylate Derivatives:

Octocrylene, sold in particular under the trade name “Uvinul N539” by BASF,
Etocrylene, sold in particular under the trade name “Uvinul N35” by BASF,

### Benzylidenecamphor Derivatives:

3-Benzylidene Camphor, manufactured under the name “Mexoryl SD” by Chimex,

Methylbenzylidene Camphor, sold under the name “Eusolex 6300” by Merck,
Polyacrylamidomethyl Benzylidene Camphor, manufactured under the name “Mexoryl SW” by Chimex,

### Triazine Derivatives:

Ethylhexyl triazone, sold in particular under the trade name “Uvinul T150” by BASF,
Diethylhexyl Butamido Triazone, sold under the trade name “Uvasorb HEB” by Sigma 3V,
2,4,6-Tris(dineopentyl 4′-aminobenzalmalonate)-s-triazine,
2,4,6-Tris(diisobutyl 4′-aminobenzalmalonate)-s-triazine,
2,4-Bis(dineopentyl 4′-aminobenzalmalonate)-6-(n-butyl 4′-aminobenzoate)-s-triazine,
2,4-Bis(n-butyl 4′-aminobenzoate)-6-(aminopropyl-trisiloxane)-s-triazine,

### Imidazoline Derivatives:

Ethylhexyl Dimethoxybenzylidene Dioxoimidazoline Propionate,

### Benzalmalonate Derivatives:

Polyorganosiloxanes comprising a benzalmalonate functional group, such as Polysilicone-15, sold under the trade name “Parsol SLX” by Hoffmann-La Roche, Dineopentyl 4′-methoxybenzalmalonate,

### Merocyanine Derivatives:

Octyl 5-N,N-diethylamino-2-phenylsulphonyl-2,4-pentadienoate.

Mention may be made, among lipophilic broad-spectrum screening agents capable of absorbing UV-A and UV-B radiation, of:

### Benzophenone Derivatives:

Benzophenone-1, sold under the trade name “Uvinul 400” by BASF,
Benzophenone-2, sold under the trade name “Uvinul D50” by BASF,
Benzophenone-3 or Oxybenzone, sold under the trade name “Uvinul M40” by BASF,
Benzophenone-6, sold under the trade name “Helisorb 11” by Norquay,
Benzophenone-8, sold under the trade name “Spectra-Sorb UV-24” by American Cyanamid,
Benzophenone-10,
Benzophenone-11,
Benzophenone-12,

### Benzotriazole Derivatives:

Drometrizole Trisiloxane, sold under the name “Silatrizole” by Rhodia Chimie,

Bumetrizole, sold under the name “Tinoguard AS” by Ciba-Geigy,

### Bisresorcinyltriazine Derivatives:

Bisethylhexyloxyphenol Methoxyphenyl Triazine, sold under the trade name “Tinosorb S” by Ciba Geigy,

### Benzoxazole Derivatives:

2,4-Bis[5-(dimethylpropyl)benzoxazol-2-yl-(4-phenyl)imino]-6-(2-ethylhexyl)imino-1,3,5-triazine, sold under the name of Uvasorb K2A by Sigma 3V.

The derivatives of the family of the diphenylbutadienemalonates or -malonitriles are the derivatives of general formula (IV):

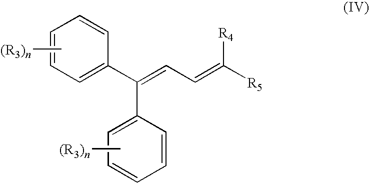

in which R₃ represents a C₁-C₂ alkyl group or a C₁-C₂ alkoxy group and n is equal to 0, 1 or 2;

R₄ and R₅, which are identical or different, represent —COOR₆, —(C=0)NHR₆, —(C═O)R₆ or —CN in which R₆ represents a linear or branched alkyl group which comprises from 1 to 12 carbon atoms and which can comprise silane, siloxane or polysiloxane groups.

Mention may in particular be made, among the diphenylbutadienemalonate or -malonitrile derivatives, without implying limitation, of:
dimethyl 2-(3,3-diphenylprop-2-enylidene)malonate
diisobutyl 2-(3,3-diphenylprop-2-enylidene)malonate
bis(1,3-dimethylbutyl) 2-(3,3-diphenylprop-2-enylidene)malonate
dineopentyl 2-(3,3-diphenylprop-2-enylidene)malonate
methyl (2Z)-2-cyano-5,5-diphenylpenta-2,4-dienoate
ethyl (trimethylsilyl)methyl (2Z)-2-(3,3-diphenyl-prop-2-enylidene)malonate
(2E)-2-cyano-5,5-diphenyl-N-(3-{1,3,3,3-tetramethyl-1-[(trimethylsilyl)oxy]disiloxanyl}propyl)penta-2,4-dienamide
ethyl 2-methyl-3-{(1,3,3,3-tetramethyl-1-[(trimethyl-silyl)oxy]disiloxanyl}propyl (2E)-2-(3,3-diphenylprop-2-enylidene)malonate
ethyl (2Z)-5,5-diphenyl-2-{[(3-{1,3,3,3-tetramethyl-1-[(trimethylsilyl)oxy]disiloxanyl}propyl)amino]-carbonyl}penta-2,4-dienoate.

Use will in particular be made, among the diphenylbutadiene derivatives mentioned above, of dineopentyl 2-(3,3-diphenylprop-2-enylidene)malonate, corresponding to the following formula:

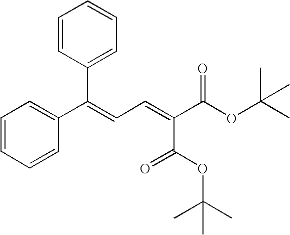

It is known to use these diphenylbutadiene derivatives in antisun compositions; Patent EP 0 916 335 describes carbon derivatives and their methods of preparation, and Patents EP 1 535 947 and EP 1 535 925 describe siloxane and silane derivatives respectively.

The derivatives of the family of the chalcones are the derivatives of following general formula (V):

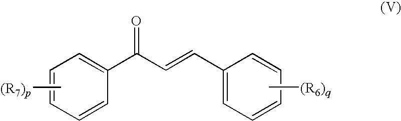

in which the R₆ and R₇ radicals denote, independently of each other, a hydrogen atom, the hydroxyl radical, a linear or branched C₁-C₁₂ alkyl or alkenyl group, a linear or branched C₁-C₁₂ alkoxy group or a linear or branched C₂-C₂₀ acyloxy group; p and q

Mention may in particular be made, among the chalcone derivatives, without implied limitation, of:
2′-hydroxychalcone
4′-hydroxychalcone
4′-methoxychalcone
2′-hydroxy-4-methoxychalcone
2′-hydroxy-4-hexyloxychalcone
2′-hydroxy-4-methylchalcone
2′-hydroxy-3-hexyloxychalcone
2′-hydroxy-4′-hexyloxy-4-methylchalcone
2′-hydroxy-4′-hexanoyloxy-4-methoxychalcone
2′,4′,4-trihydroxy-3,3′-diallylchalcone (known under the name of Kazonol)
2′,4′,4-trihydroxy-5′-(3-methylbut-2-enyl)chalcone (known under the name of Broussochalcone B)
2′,3′,4′,6′,4-pentahydroxychalcone (known under the name of Carthamin).

Use will in particular be made, among the chalcone derivatives mentioned above, of 4′-hydroxychalcone, corresponding to the following formula (Va):

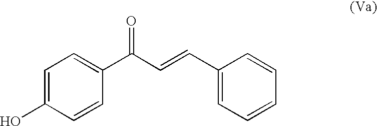

or 2′,3′,4′,6′,4-pentahydroxychalcone (known under the name of Carthamin), corresponding to the following formula (Vb):

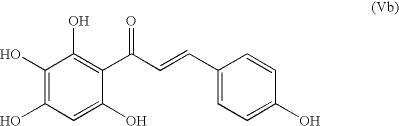

It is known to use these chalcone derivatives in antisun compositions, in particular in Patents FR 2 555 167, FR 2 602 228 and FR 2 608 150.

The lipophilic organic UV screening agent can preferably be chosen from:

salicylic derivatives, in particular homosalate or ethylhexyl salicylate,

cinnamic derivatives, such as ethylhexyl methoxycinnamate,

β,β-diphenylacrylate derivatives, such as octocrylene,

dibenzoylmethane derivatives, such as butyl methoxydibenzoylmethane,

triazine derivatives, such as ethylhexyl triazone or diethylhexyl butamido triazone,

benzotriazole derivatives, such as drometrizole trisiloxane,

and their mixtures.

Mention may be made, among the hydrophilic or water-soluble UV screening agents which can be used according to the invention, of the following screening agents, denoted below under their INCI names: water-soluble UV-A screening agents capable of absorbing UV radiation from 320 to 400 nm, such as: Terephthalylidene Dicamphor Sulphonic Acid, manufactured under the name “Mexoryl SX” by Chimex, bisbenzoxazolyl derivatives, such as described in Patent, EP 669 323 and U.S. Pat. No. 2,463,264, more particularly the compound Disodium Phenyl Dibenzimidazole Tetrasulphonate, sold under the trade name “Neo Heliopan AP” by Haarmann and Reimer,

water-soluble UV-B screening agents capable of absorbing UV radiation from 280 to 320 nm, such as: p-aminobenzoic (PABA) derivatives, such as PABA, Glyceryl PABA and PEG-25 PABA, sold under the name “Uvinul P25” by BASF,

Phenylbenzimidazole sulphonic acid, sold in particular under the trade name “Eusolex 232” by Merck,

ferulic acid,

salicylic acid,

DEA methoxycinnamate,

Benzylidene Camphor Sulphonic Acid, manufactured under the name “Mexoryl SL” by Chimex,

Camphor Benzalkonium Methosulphate, manufactured under the name “Mexoryl SO” by Chimex, and water-soluble UV-A and UV-B screening agents, such as: Benzophenone-4, sold under the trade name “Uvinul MS40” by BASF,

Benzophenone-5, and

Benzophenone-9.

### Aqueous Phase

The aqueous phase of the compositions according to the invention comprises at least water. According to the formulation form of the composition, the amount of aqueous phase can range from 0.1 to 99% by weight, preferably from 0.5 to 98% by weight, better still from 30 to 95% by weight and even better still from 40 to 95% by weight, with respect to the total weight of the composition. This amount depends on the formulation form of the composition desired. The amount of water can represent all or a portion of the aqueous phase and it is generally at least 30% by weight, with respect to the total weight of the composition.

The aqueous phase can comprise at least one hydrophilic solvent, such as, for example, substantially linear or branched lower monoalcohols having from 1 to 8 carbon atoms, such as ethanol, propanol, butanol, isopropanol or isobutanol, polyols, such as propylene glycol, isoprene glycol, butylene glycol, propylene glycol, glycerol, sorbitol, polyethylene glycols and their derivatives, and their mixtures.

### Fatty Phase

The proportion of the fatty phase of the emulsion can range, for example, from 1 to 80% by weight, preferably from 2 to 50% by weight and better still from 5 to 30% by weight, with respect to the total weight of the composition.

This amount indicated does not comprise the content of lipophilic screening agents.

The nature of the fatty phase (or oily phase) of the emulsion is not critical. The fatty phase can thus be composed of any fatty substance conventionally used in the cosmetic or dermatological fields; it comprises in particular at least one oil (fatty substance which is liquid at 25° C.)

Mention may be made, as oils which can be used in the composition of the invention, for example, of:

hydrocarbon oils of animal origin, such as perhydrosqualene;

hydrocarbon oils of vegetable origin, such as liquid triglycerides of fatty acids comprising from 4 to 10 carbon atoms, such as triglycerides of heptanoic or octanoic acids, or also, for example, sunflower, maize, soyabean, cucumber, grapeseed, sesame, hazelnut, apricot, macadamia, arara, castor or avodaco oils, triglycerides of caprylic/capric acids, such as those sold by Stearineries Dubois or those sold under the names Miglyol 810, 812 and 818 by Dynamit Nobel, jojoba oil or shea butter oil;

synthetic esters and ethers, in particular of fatty acids, such as oils or formulae R^{a}COOR^{b} and R^{a}OR^{b} in which R^{a} represents the residue of a fatty acid comprising from 8 to 29 carbon atoms, and R^{b} represents a branched or unbranched hydrocarbon chain comprising from 3 to 30 carbon atoms, such as, for example, Purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentraerythrityl tetraisostearate;

substantially linear or branched hydrocarbons of mineral or synthetic origin, such as liquid paraffins, which may or may not be volatile, and their derivatives, petrolatum, polydecenes, isohexadecane, isododecane or hydrogenated polyisobutene, such as Parleam® oil;

fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;

alkoxylated fatty alcohols and in particular ethoxylated fatty alcohols, such as oleth-12, ceteareth-12 and ceteareth-20;

fluorinated oils which partially comprise hydrocarbon and/or silicone, such as those described in the document JP-A-2-295912. Mention may also be made, as fluorinated oils, of perfluoromethylcyclopentane and perfluoro-1,3-dimethylcyclohexane, which are sold under the names of “Flutec PC1®” and “Flutec PC3®” by BNFL Fluorochemicals; perfluoro-1,2-dimethylcyclobutane; perfluoroalkanes, such as dodecafluoropentane and tetradecafluorohexane, which are sold under the names of “PF 5050®” and “PF 5060®” by 3M, or also bromoperfluorooctyl, sold under the name “Foralkyle” by Atochem; nonafluoromethoxybutane, sold under the name “MSX 4518®” by 3M, and nonafluoroethoxyisobutane; or perfluoromorpholine derivatives, such as 4-(trifluoromethyl)perfluoromorpholine, sold under the name “PF 5052®” by 3M;

silicone oils, such as volatile or non-volatile polymethylsiloxanes (PDMS) comprising a substantially linear or cyclic silicone chain which are liquid or pasty at ambient temperature, in particular cyclopolydimethylsiloxanes (cyclomethicones), such as cyclohexadimethylsiloxane and cyclopentadimethyl-siloxane; polydimethylsiloxanes comprising pendant alkyl, alkoxy or phenyl groups or alkyl, alkoxy or phenyl groups at the end of the silicone chain, which groups have from 2 to 24 carbon atoms; or phenylated silicones, such as phenyl trimethicones, phenyl dimethicones, phenyl(trimethylsiloxy)diphenylsiloxanes, diphenyl dimethicones, diphenyl(methyldiphenyl)-trisiloxanes, (2-phenylethyl)trimethylsiloxysilicates and polymethylphenylsiloxanes;

their mixtures.

The term “hydrocarbon oil” in the list of the oils mentioned above is understood to mean any oil comprising predominantly carbon and hydrogen atoms and optionally ester, ether, fluorinated, carboxylic acid and/or alcohol groups.

The other fatty substances which can be present in the oily phase are, for example, fatty acids comprising from 8 to 30 carbon atoms, such as stearic acid, lauric acid, palmitic acid and oleic acid; waxes, such as lanolin, beeswax, carnauba or candelilla wax, paraffin, lignite or microcrystalline waxes, ceresin or ozokerite, or synthetic waxes, such as polyethylene waxes or Fischer-Tropsch waxes; or petrolatum paste.

These fatty substances can be chosen in a way varied by a person skilled in the art so as to prepare a composition having the desired properties, for example of consistency or texture.

The emulsions generally comprise at least one emulsifier chosen from amphoteric, anionic, cationic or nonionic emulsifiers, used alone or as a mixture. The emulsifiers are appropriately chosen according to the emulsion to be obtained (W/O or O/W).

The emulsifiers are generally present in the composition in a proportion ranging from 0.1 to 30% by weight and preferably from 0.2 to 20% by weight, with respect to the total weight of the composition.

Mention may be made, for the W/0 emulsions, for example, as emulsifiers, of dimethicone copolyols, such as the mixture of cyclomethicone and of dimethicone copolyol sold under the name “DC 5225 C”, by Dow Corning, and alkyl dimethicone copolyols, such as lauryl dimethicone copolyol, sold under the name “Dow Corning 5200 Formulation Aid” by Dow Corning, cetyl dimethicone copolyol, sold under the name Abil EM 90® by Goldschmidt, or the polyglyceryl-4 isostearate/cetyl dimethicone copolyol/hexyl laurate mixture sold under the name Abil WE 09 by Goldschmidt. It is also possible to add one or more coemulsifiers thereto. Advantageously, the coemulsifier can be chosen from the group consisting of polyol alkyl esters. Mention may in particular be made, as polyol alkyl esters, of glycerol and/or sorbitan esters, for example polyglycerol isostearate, such as the product sold under the name Isolan GI 34 by Goldschmidt, sorbitan isostearate, such as the product sold under the name Arlacel 987 by ICI, or sorbitan glyceryl isostearate, such as the product sold under the name Arlacel 986 by ICI, and their mixtures.

For the O/W emulsions, mention may be made, for example, as emulsifiers, of nonionic surfactants and in particular esters of polyols and of a fatty acid possessing a saturated or unsaturated chain comprising, for example, from 8 to 24 carbon atoms and better still from 12 to 22 carbon atoms, and their oxyalkylenated derivatives, that is to say comprising oxyethylene and/or oxypropylene units, such as esters of glycerol and of a C₈-C₂₄ fatty acid, and their oxyalkylenated derivatives; esters of polyethylene glycol and of a C₈-C₂₄ fatty acid, and their oxyalkylenated derivatives; esters of sorbitol and of a C₈-C₂₄ fatty acid, and their oxyalkylenated derivatives; ethers of fatty alcohols; ethers of a sugar and of C₈-C₂₄ fatty alcohols, and their mixtures.

Mention may in particular be made, as ester of glycerol and of a fatty acid, of glyceryl stearate (glyceryl mono-, di- and/or tristearate) (CTFA name: glyceryl stearate) or glyceryl ricinoleate, and their mixtures.

Mention may in particular be made, as ester of polyethylene glycol and of a fatty acid, of polyethylene glycol stearate (polyethylene glycol mono-, di- and/or tristearate) and more especially of polyethylene glycol 50 EO monostearate (CTFA name: PEG-50 stearate), polyethylene glycol 100 EO monostearate (CTFA name: PEG-100 stearate) and their mixtures.

Use may also be made of mixtures of these surfactants, such as, for example, the product comprising glyceryl stearate and PEG-100 stearate sold under the name Arlacel 165 by Uniquema and the product comprising glyceryl stearate (glyceryl mono/distearate) and potassium stearate sold under the name Tegin by Goldschmidt (CTFA name: glyceryl stearate SE).

Mention may be made, as ethers of fatty alcohols, for example, of ethers of polyethylene glycol and of a fatty alcohol comprising from 8 to 30 carbon atoms and in particular from 10 to 22 carbon atoms, such as ethers of polyethylene glycol and of cetyl alcohol, stearyl alcohol or cetearyl alcohol (mixture of cetyl alcohol and stearyl alcohol). Mention may be made, for example, of ethers comprising from 1 to 200 and preferably from 2 to 100 oxyethylene groups, such as those bearing the CTFA names ceteareth-20 and ceteareth-30, and their mixtures.

According to a specific embodiment, the composition comprises at least one emulsifier chosen from hydrophobic modified inulins.

The term “hydrophobic modified inulin” according to the invention is understood to mean in particular an inulin modified by hydrophobic chains, in particular modified by grafting hydrophobic chains to the hydrophilic backbone of the inulin.

Inulin belongs to the family of essentially linear fructans, the fructose units of which are predominantly connected via β-2-1 bonds.

Inulin can be obtained, for example, from chicory, dahlia or Jerusalem artichoke. Preferably, the inulin used in the composition according to the invention is obtained, for example, from chicory.

The inulins used in the compositions according to the invention are hydrophobically modified. In particular, they are obtained by grafting hydrophobic chains to the hydrophilic backbone of the fructan.

The hydrophobic chains capable of being grafted to the main chain of the fructan can in particular be saturated or unsaturated and linear or branched hydrocarbon chains having from 1 to 50 carbon atoms, such as alkyl, arylalkyl, alkylaryl or alkylene groups, divalent cycloaliphatic groups or organopolysiloxane chains. These hydrocarbon or organopolysiloxane chains can in particular comprise one or more ester, amide, urethane, carbamate, thiocarbamate, urea, thiourea and/or sulphonamide functional groups, such as, in particular, methylenedicylohexyl and isophorone, or divalent aromatic groups, such as phenylene.

In particular, the inulin exhibits a degree of polymerisation of 2 to approximately 1000 and preferably of 2 to approximately 60 and a degree of substitution of less than 2, based on one fructose unit.

According to a preferred embodiment, the hydrophobic chains exhibit at least one alkyl carbamate group of formula R—NH—CO— in which R is an alkyl group having from 1 to 22 carbon atoms.

According to a more preferred embodiment, the hydrophobic chains are lauryl carbamate groups.

In particular, mention may be made, by way of illustration and without implied limitation of the hydrophobic modified inulins which can be used in the compositions according to the invention, of stearoyl inulin, such as those sold under the names Lifidrem INST by Engelhard and Rheopearl INS by Ciba, palmitoyl inulin, undecylenoyl inulin, such as those sold under the names Lifidrem INUK and Lifidrem INUM by Engelhard, and inulin lauryl carbamate, such as that sold under the name Inutec SP1 by Orafti.

In particular, use is made of an inulin grafted with lauryl carbamate, in particular resulting from the reaction of lauryl isocyanate with an inulin, in particular resulting from chicory. Mention may in particular be made, as example of these compounds, of the product sold under the name Inutec SP1 by Orafti. The level of hydrophobic modified inulin in the composition of the invention can range from 0.01 to 20% by weight, preferably from 0.01 to 10% by weight, preferably from 0.05 to 10% by weight, in particular from 0.1 to 10% by weight, preferably from 0.1 to 5% by weight and more preferably still from 0.1 to 1% by weight (of active material), with respect to the total weight of the composition.

All the compositions of the invention can comprise one or more of the adjuvants normal in the cosmetic and dermatological fields, such as hydrophilic or lipophilic gelling and/or thickening agents; moisturizing agents; emollients; hydrophilic or lipophilic active principles, agents for combating free radicals; sequestering agents; antioxidants; preservatives; basifying or acidifying agents; fragrances; film-forming agents; colouring materials (pigments, such as iron oxides and titanium dioxide, pearlescent agents or soluble dyes); fillers; and their mixtures.

The amounts of these various adjuvants are those conventionally used in the fields under consideration. In particular, the amounts of active principles vary according to the desired aim andare those conventionally used in the fields under consideration, for example from 0.1 to 20% and preferably from 0.5 to 10% of the total weight of the composition.

Mention may be made, as hydrophilic gelling agents other than the polymers described above, for example, of carboxyvinyl polymers, such as carbopols (carbomers), and Pemulen products (acrylate/C₁₀-C₃₀-alkyl acrylate copolymer); polyacrylamides, such as, for example, the crosslinked copolymers sold under the names Sepigel 305 (CTFA name: polyacrylamide/C13-14 isoparaffin/laureth-7) or Simulgel 600 (CTFA name: acrylamide/sodium acryloyldimethyltaurate copolymer/isohexadecane/polysorbate 80) by Seppic; cellulose derivatives, such as hydroxyethylcellulose; polysaccharides and in particular gums, such as xanthan gum; and their mixtures.

Mention may be made, as lipophilic gelling agents, of modified clays, such as hectorite and its derivatives, for example the products sold under the Bentone names.

### Active Principles

As indicated above, the composition of the invention is stable in the presence of oxidation-sensitive hydrophilic active principles and makes it possible to stabilize said active principles. According to the invention, the term “hydrophilic active principle” is understood to mean a compound having a solubility in water of at least 0.25% at ambient temperature (25° C.). In addition, according to the invention, the term “oxidation-sensitive hydrophilic active principle” is understood to mean any active principle of natural or synthetic origin capable of undergoing decomposition by an oxidation mechanism. This oxidation phenomenon can have several causes, in particular the presence of oxygen, of light or of metal ions, a high temperature or also some pH conditions.

Mention may be made, as examples of oxidation-sensitive hydrophilic active principle, without implied limitation, of ascorbic acid and its derivatives, such as 5,6-di-O-dimethylsilylascorbate (sold by Exsymol under the reference Pro-AA), the potassium salt of dl-α-tocopheryl-21-ascorbyl phosphate (sold by Senju Pharmaceutical under the reference Sepivital EPC), magnesium ascorbyl phosphate or sodium ascorbyl phosphate (sold by Roche under the reference Stay-C 50), phloroglucinol, enzymes and their mixtures. Use may be made, according to a preferred embodiment of the invention, among oxidation-sensitive hydrophilic active principles, of ascorbic acid. The ascorbic acid can be of any nature. Thus, it can be of natural origin, in the form of a powder or in the form of an orange juice, preferably a concentrated orange juice. It can also be of synthetic origin, preferably in the form of a powder.

Mention may be made, as other active principles which can be used in the composition of the invention, for example, of moisturizing agents, such as protein hydrolysates and polyols, such as glycerol or glycols, for example polyethylene glycol; natural extracts; anti-inflammatories; procyanidol oligomers; vitamins, such as vitamin A (retinol), vitamin E (tocopherol), vitamin B5 (panthenol), vitamin B3 (niacinamide), the derivatives of these vitamins (in particular esters) and their mixtures; urea; caffeine; depigmenting agents, such as kojic acid, hydroquinone and caffeic acid; salicyclic acid and its derivatives; α-hydroxy acids, such as lactic acid and glycolic acid and their derivatives; retinoids, such as carotenoids and vitamin A derivatives; hydrocortisone; melatonin; algal, fungal, plant, yeast or bacterial extracts; steroids; antibacterial active principles, such as 2,4,4′-trichloro-2′-hydroxydiphenyl ether (or triclosan), 3,4,4′-trichlorocarbanilide (or triclocarban) and the acids indicated above and in particular salicylic acid and its derivatives; matifying agents, such as fibres; tightening agents; and their mixtures.

In addition to organic sunscreens (or UV screening agents), the composition according to the invention can additionally comprise inorganic UV screening agents which are metal oxide particles having a mean individual particle size of less than or equal to 500 nm, more preferably between 5 nm and 500 nm, more preferably still of between 10 nm and 100 nm and preferably of between 15 nm and 50 nm. They can in particular be chosen from titanium, zinc, iron, zirconium or cerium oxides or their mixtures. According to their more or less pronounced lipophilic nature or, on the contrary, hydrophilic nature, the inorganic screening agents can be present either in the fatty phase of the emulsion or in the aqueous phase or even in both phases simultaneously.

The compositions in accordance with the present invention can additionally comprise insoluble organic UV screening agents comprising at least one group which absorbs UV radiation, which screening agents can be chosen in particular from insoluble organic UV screening agents of oxalanilide, vinyl amide, cinnamamide, benzazole, benzofuran, arylvinylidene ketone, acrylonitrile amide, acrylonitrile sulphonamide, acrylonitrile carbamate or phenylenebis(benzoxazinone) type.

Mention may be made, as fillers which can be used in the composition of the invention, for example, of pigments, such as titanium, zinc or iron oxides and organic pigments; kaolin; silica; talc; boron nitride; spherical organic powders; fibres; and their mixtures. Mention may be made, as spherical organic powders, for example, of polyamide powders and in particular Nylon®, such as Nylon-1 or Polyamide 12, powders sold under the Orgasol names by Atochem; polyethylene powders; Teflon®; microspheres based on acrylic copolymers, such as those made of ethylene glycol dimethacrylate/lauryl methacrylate copolymer sold by Dow Corning under the name Polytrap; expanded powders, such as hollow microspheres and in particular the microspheres sold under the name Expancel by Kemanord Plast or under the name Micropearl F 80 ED by Matsumoto; silicone resin microbeads, such as those sold under the name Tospearl by Toshiba Silicone; polymethyl methacrylate microspheres, sold under the name Microsphere M-100 by Matsumoto or under the name Covabead LH85 by Wackherr; ethylene/acrylate copolymer powders, such as those sold under the name Flobeads by Sumitomo Seika Chemicals; or powders formed from natural organic materials, such as starch powders, in particular powders formed from crosslinked or noncrosslinked maize, wheat or rice starches, such as the powders formed from starch crosslinked with octenyl succinic anhydride sold under the name Dry-Flo by National Starch. Mention may be made, as fibres, for example, of polyamide fibres, such as in particular fibres formed from Nylon 6 (or Polyamide 6) (INCI name: Nylon 6) or from Nylon 6,6 (or Polyamide 66) (INCI name: Nylon 66) or such as fibres formed from poly(p-phenylene terephthalamide); and their mixtures. These fillers can be present in amounts ranging from 0 to 20% by weight and preferably from 0.5 to 10% by weight, with respect to the total weight of the composition.

Of course, a person skilled in the art will take care to choose the optional adjuvant or adjuvants added to the composition according to the invention so that the advantageous properties intrinsically attached to the composition in accordance with the invention are not, or not substantially, detrimentally affected by the envisaged addition.

The compositions according to the invention can be provided in the form of dispersions of the lotion or serum type, of emulsions with a liquid or semi-liquid consistency of the milk type, obtained by dispersion of a fatty phase in an aqueous phase (O/W) or vice versa (W/O), of suspensions or emulsions with a soft, semi-solid or solid consistency of the cream or gel type, of multiple (W/O/W or O/W/O) emulsions, of microemulsions, of vesicular dispersions of ionic and/or nonionic type, or of wax/aqueous phase dispersions. These compositions are prepared according to the usual methods.

According to a preferred embodiment of the invention, the composition is provided in the form of an emulsion and in particular of an O/W emulsion.

In addition, the compositions used according to the invention can be more or less fluid and can have the appearance of a gel, of a white or coloured cream, of an ointment, of a milk, of a lotion, of a serum, of a paste or of a foam.

The composition preferably exhibits a pH which respects the skin and which generally ranges from 3 to 8 and preferably from 4.5 to 7.

Another subject-matter of the invention is the cosmetic use of a cosmetic composition as defined above as sun protection product (protection against the sun and/or the UV radiation of tanning devices).

The examples which follow will make possible a better understanding of the invention without, however, exhibiting a limiting nature. The amounts shown are as % by weight, unless otherwise mentioned.

### Examples 1 to 3

### Moisturizing O/W Emulsions SPF 15

A composition according to the invention (Example 1) comprising a lipophilic screening agent and a superabsorbent polymer and two comparative compositions (Examples 2 and 3) comprising a lipophilic sunscreen and acrylic polymers which are not superabsorbent are prepared:

| | | Ex. 1 | Ex. 2 | Ex. 3 |
|---|---|---|---|---|
| Phase | | (inventtion) | (comp.) | (comp.) |
| A | Water | q.s. for | q.s. | q.s. |
| | | 100 | for 100 | for 100 |
| | Preservatives | q.s. | q.s. | q.s. |
| B | Isononyl isononanoate | 8 | 8 | 8 |
| | Butyl Methoxydibenzoyl- | 3 | 3 | 3 |
| | methane (“Parsol 1789” from | | | |
| | Hoffman-La Roche) | | | |
| | Octocrylene (“Uvinul N539” | 7 | 7 | 7 |
| | from BASF) | | | |
| | Ethylhexyl Salicylate (“Neo | 5 | 5 | 5 |
| | Heliopan OS” from Symrise) | | | |
| | Inulin lauryl carbamate | 0.3 | 0.3 | 0.3 |
| | (Inutec SP1 from Orafti) | | | |
| C | Glycerol | 3 | 3 | 3 |
| | Microspheres formed of | 0.5 | — | — |
| | crosslinked polyacrylates at | | | |
| | 89% as active materials as a | | | |
| | mixture with silica in water | | | |
| | (Aqua Keep 10 SH NF from | | | |
| | Sumitomo Seika) | | | |
| | Crosslinked sodium | — | — | 1 |
| | polyacrylate at 90% as dry | | | |
| | matter in water (Cosmedia SP | | | |
| | from Cognis) | | | |
| | Acrylic homopolymer at 98% | | 0.5 | |
| | as dry matter in water | | | |
| | (Carbopol 980 from Lubrizol) | | | |
| | Sodium hydroxide | | 0.2 | |

### Procedure

Phase A is heated up to 85° C. and then brought back to 75° C.

Phase B is added to phase A at 75° C. using a mixer.

Phase C is added to the mixture (A+B) while stirring with a Rayneri mixer and then allowed to cool to ambient temperature while stirring with a Rayneri mixer.

Three cream gels with comparable viscosities are obtained.

The composition of Example 1 according to the invention is soft and fresh on application without any tacky effect, either during application or after the penetration of the product into the skin, in contrast to the compositions of Examples 2 and 3, which leave a greasy and tacky film on the skin.

### Examples 4 and 5

### Antisun O/W Emulsions SPF 30

A composition according to the invention (Example 4) comprising a lipophilic screening agent and a superabsorbent polymer and a comparative composition (Example 5) comprising a lipophilic sunscreen and an acrylic polymer which is not superabsorbent are prepared:

| | | Example 4 | Example 5 |
|---|---|---|---|
| Phase | | (invention) | (comparative) |
| A | Water | q.s. for | 56.64 |
| | | 100 | |
| | Propylene glycol | 6 | 6 |
| | Glycerol | 6 | 6 |
| | Potassium cetyl phosphate | 1 | 1 |
| | (Amphisol K from DSM | | |
| | Nutritional Products) | | |
| | EDTA | 0.1 | 0.1 |
| | β,β′-Camphosulphonic acid | 0.9 | 0.9 |
| | [1,4-divinylbenzene] as | | |
| | an aqueous solution | | |
| | comprising 33% of active | | |
| | materials (Mexoryl SX | | |
| | from Chimex) | | |
| | Triethanolamine | 0.16 | 0.16 |
| | Preservatives | q.s. | q.s. |
| B | Stearic acid | 1 | 1 |
| | Glyceryl mono/distearate | 1 | 1 |
| | and polyethylene glycol | | |
| | stearate (100 EO) mixture | | |
| | (Arlacel 165 FL from | | |
| | Croda) | | |
| | C₁₂₋₁₅ Alkyl benzoate | 7.5 | 7.5 |
| | (Tegosoft TN from Evonik | | |
| | Goldschmidt) | | |
| | Butyl Methoxydibenzoyl- | 3 | 3 |
| | methane (“Parsol 1789” | | |
| | from Hoffmann-La Roche) | | |
| | Octocrylene (Uvinul N539 | 7 | 7 |
| | from BASF) | | |
| | Ethylhexyl triazone | 0.5 | 0.5 |
| | (Uvinul T150 from BASF) | | |
| | Tocopherols (Decanox MTS | 0.1 | 0.1 |
| | 50 ADM) | | |
| | Triethanolamine | 0.3 | 0.3 |
| C | Rutile titanium oxide | 3 | 3 |
| | treated with aluminium | | |
| | stearate/alumina (Micro | | |
| | Titanium Dioxide MT-100 | | |
| | T V from Tayca) | | |
| D | Isohexadecane | 2 | 2 |
| | Microspheres formed of | 0.5 | — |
| | crosslinked polyacrylates | | |
| | at 89% as active | | |
| | materials as a mixture | | |
| | with silica in water | | |
| | (Aqua Keep 10 SH NF from | | |
| | Sumitomo Seika) | | |
| | Acrylic homopolymer at | — | 0.5 |
| | 98% as dry matter in | | |
| | water (Carbopol 980 from | | |
| | Lubrizol) | | |
| | Sodium hydroxide | | 0.2 |
| | Xanthan gum | 0.1 | 0.1 |
| E | Ethanol | 2 | 2 |

### Procedure

Phase A is heated up to 85° C. and then brought back to 75° C.

Phase B is added to phase A at 75° C. using a mixer. While stirring with a Rayneri mixer, phase C is dispersed in the mixture (A+B), phase D is then added and then phase E is added, still with stirring, and then cooling is allowed to take place down to ambient temperature while stirring with a Rayneri mixer.

The composition of Example 4 according to the invention is soft and fresh on application without any tacky effect, either during application or after the penetration of the product into the skin, in contrast to the composition of Example 5, which leaves a sticky feel on application to the skin and a tacky film after penetration.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition in the form of an emulsion comprising an aqueous phase, a fatty phase, a superabsorbent polymer, and an organic UV screening agent.

2. The composition according to claim 1, wherein the superabsorbent polymer is provided in the form of particles having a number-average diameter ranging from 10 μm to 1000 μm.

3. The composition according to claim 1, wherein the superabsorbent polymer is provided in the form of particles having a number-average diameter of less than or equal to 100 μm.

4. The composition according to claim 1, wherein the superabsorbent polymer exhibits a water-absorbing capacity of 10 to 100 g/g.

5. The composition according to claim 1, wherein the superabsorbent polymer is chosen from crosslinked sodium polyacrylates, starches grafted by an acrylic polymer, hydrolysed starches grafted by an acrylic polymer, polymers based on starch, on gum and on cellulose derivative, and their blends.

6. The composition according to claim 1, wherein the superabsorbent polymer is chosen from crosslinked acrylic homo- or copolymers which are optionally neutralized.

7. The composition according to claim 1, wherein the superabsorbent polymer is chosen from crosslinked sodium polyacrylates.

8. The composition according to claim 1, wherein the superabsorbent polymer is provided in the form of spherical particles.

9. The composition according to claim 1, wherein the superabsorbent polymer is present in a content of 0.03 to 15% by weight with respect to the total weight of the composition.

10. The composition according to claim 1, comprising a lipophilic organic UV screening agent.

11. The composition according to claim 1, wherein the organic UV screening agent is chosen from para-aminobenzoic acid derivatives, salicylic derivatives, cinnamic derivatives, benzophenones or aminobenzophenones, anthranilic derivatives, dibenzoylmethane derivatives, β,β-diphenylacrylate derivatives, benzylidenecamphor derivatives, phenylbenzimidazole derivatives, benzotriazole derivatives, triazine derivatives, bisresorcinyltriazines, imidazoline derivatives, benzalmalonate derivatives, 4,4-diarylbutadiene derivatives, benzoxazole derivatives, merocyanines, diphenylbutadienemalonate or -malonitrile derivatives, chalcones and their mixtures.

12. The composition according to claim 1, wherein the organic UV screening agent is chosen from:
salicylic derivatives,
cinnamic derivatives,
β,β-diphenylacrylate derivatives,
dibenzoylmethane derivatives,
triazine derivatives,
benzotriazole derivatives,
and their mixtures.

13. The composition according to claim 1, comprising a hydrophilic organic UV screening agent.

14. The composition according to claim 1, wherein the total amount of organic UV screening agent(s) is 0.05 to 30% by weight with respect to the total weight of the composition.

15. A method for the treatment of a keratinous substance, comprising applying the composition of claim 1 to the keratinous substance.
